(19) United States
(12) Patent Application Publication
(10) Pub. No.: US 2022/0184204 A1
(43) Pub. Date: Jun. 16, 2022

(54) Self-assembled protein nanoparticle and its applications thereof

(71) Applicant: Ming-Chung Kan, Taipei (TW)

(72) Inventor: Ming-Chung Kan, Taipei (TW)

(21) Appl. No.: 17/472,721

(22) Filed: Sep. 13, 2021

Related U.S. Application Data
(60) Provisional application No. 63/077,769, filed on Sep. 14, 2020.

(51) Int. Cl. A61K 39/155; A61K 39/02; A61K 47/69; A61P 11/00; A61P 31/14; C07K 14/00; C12N 7/00

(52) U.S. Cl. CPC A61K 39/155; A61K 39/02; A61K 47/6931; A61P 11/00; C12N 2760/18034; C07K 14/00; C12N 7/00; C07K 2319/735; C12N 2760/18022; A61P 31/14

(57) ABSTRACT

Self-assembled protein nanoparticle (SAPN) are excellent antigen due to its ability to simultaneously present multiple epitopes to B cell and generate much stronger B cell receptor signaling than single epitope. Most of the SAPN are derived from capsid protein of virus or bacterial phage, which suffer from low particle stability, existing antibody against capsid protein and structural intolerant to peptide insertion. In this invention, we have created a SAPN using non-viral protein that is both thermal stable and tolerate to target peptide insertions. The assembling subunit of this SAPN is a fusion protein between two components: first, a polymerization module composed of an amphipathic helical peptide modified from M2 protein of type A influenza virus and second, a target peptide presentation module that composed of a superfolder green fluorescent protein (sfGFP) with a peptide insertion site on a specific loop of sfGFP. This particle is able to incorporate target peptide through genetic recombination and presented the target protein in the surface of nanoparticle to stimulate the production of high affinity antibody against target peptide without using adjuvant.

### FIELD OF INVENTION

The present invention related to a novel hydrophobic patch free self-assembled protein nanoparticle that is used in immunology, diagnostic and disease treatment.

### DESCRIPTION OF RELEVANT ARTS

Most of the SAPN reported in literatures are derived from capsid proteins of virus or bacterial phage. These SAPN subunit proteins includes HBV surface antigen (HBsAg), Human papilloma virus L1major capsid protein (HPV L1), capsid protein from Acinetobacter phage (AP205), and core antigen of HBV (HBcAg). Two structural features of SAPNs, molecular specificity and multivalency, make them suitable vaccine adjuvants and carriers. The high antigen density and structurally ordered antigen arrangement on SAPNs resembles the recognition patterns on pathogens and thus facilitates the cross-linking of antigens with the BCRs. This multivalent interaction is a key step in provoking a potent immune response and is a solution for the weak immunogenicity of subunit vaccines. In fact, SAPNs elicit high titers of high affinity neutralizing IgG in addition to CD8+ T-cell-mediated protection. They not only trigger complement activation but also help in creating microenvironments that promote the interaction of the vaccine with APCs. Therefore, SAPNs have been used in both prophylactic and immunotherapeutic vaccines. The incorporation of antigens on self-assembled protein and subsequent production of chimeric SAPNs is accomplished through a direct self-assembly process or covalent chemical bonding of antigens to nanoparticles.

One of the best characterized viral SAPN is based on the core antigen of hepatitis B virus (HBcAg). The HBcAg monomer contains an assembly domain (1-149 aa) and a C-terminal domain (CTD) for binding of the nucleic acids. The assembly domain consists of five α-helices and the major immunodominant region (MIR), located between helix 3 and helix 4 and served as the insertion site for foreign peptide. HBcAg monomers associates into dimer and spontaneously assembled through interdimer contacts during microbial protein expression. The application of HBcAg in human vaccine design have faced two challenges. First, this SAPN is based on a human pathogen, so it will not be effective in the 450 million chronic HBV carriers worldwide due to existing antibody and may be less effective for those already exposed to the virus. Second, many of the foreign epitopes inserted into the core gene destroy the self-assembly property of the HBcAg particle. These two issues make development of a SAPN that fulfilled these two issues desireable.

Fluorescent protein is a protein family that shares similar 3D structure and functions. Fluorescent protein homologs have been identified in various species from corals, sea anemones, zoanthids, copepods and lancelets. Fluorescent proteins from different species share similar 3D structure but low similarity in primary protein sequences. Fluorescent proteins share a beta barrel structure composed of 11 beta sheets and an alpha helix containing a three amino acids chromophore running through the barrel. Each beta sheet is connected through a loop to the next one, certain loop is more tolerant to peptide insertion without affecting the structural integrity and its function to generate fluorescence. Fluorescent proteins are highly thermal stable and fast folding that enable easy fusion with another protein without disrupting structure of both proteins. Fluorescent proteins have been applied in multiple fields, like in cell biological studies that serves as a reporter to monitor target protein localization under fluorescent microscope when fused to a target protein; biochemical studies that mark the close interaction between two proteins through energy transfer between two compatible fluorescent proteins pair each fused to one target protein. The stability of fluorescent protein and its tolerance to peptide insertion can be improved by a direct evolution process that combines random mutagenesis of fluorescent protein coding region by DNA shuffling and screening for clones that fold correctly in the presence of structure disruptor. The disruptor can be a peptide sequence inserted between beta sheet 8 and 9. Through this process, fluorescent proteins from all species can be modified into a superfolder form. It has been shown by Dr. Kobayashi in Juntendo University that it is possible to insert a functional peptide into the loop between beta sheet 8 and 9 of sfGFP without disrupting structural integrity that serves as tandem affinity tag for purification. One of the peptide included in the affinity tag including a streptavidin binding peptide (Sequence: MDEKTTGWRGGHVVEGLAGELEQLRARLEHHPQGQREP), a peptide of 38 amino acids that interacts with streptavidin with high specificity and affinity. Dr. Pavoor in University of Chicago described the generation of fluorescent protein based antibody library by inserting random peptide sequence into two proximal loops in GFP. When this antibody library is used to screen against model protein, fluorescent antibody can be identified that possess nanomolar affinity.

Protein aggregation is a biological phenomenon in which intrinsically disordered proteins or mis-folded proteins interact through hydrophobic interaction. After synthesis, proteins typically fold into a particular three-dimensional conformation that is the most thermodynamically favorable: their native state. This folding process is driven by the hydrophobic effect: a tendency for hydrophobic portion of the protein to shield themselves from the hydrophillic environment of the cell by burying into the interior of the protein. Thus, the exterior of a protein is typically hydrophilic, whereas the interior is typically hydrophobic. The presence of hydrophobic patch on the surface of a protein increases the chance of forming protein aggregation through interacting with hydrophobic patch of another protein. Extended protein aggregation leads to protein precipitation and inactivation.

Amphipathic helical peptide is a structural motif that mediates protein interacting with lipid membrane. These interactions can be classified into several functions: first, mediates the membrane localization of peripheral membrane proteins; second, disrupts membrane integrity by some bacterial toxins; third, creates membrane curvature for virus budding. Amphipathic helical peptide from type A influenza virus M2 protein (M2AH) is known to mediate virus budding and membrane anchorage of M2 proton channel. The M2AH of type A influenza virus is located from amino acid 44 to amino acid 62 of M2 protein with some variations between different type A influenza viral strains. The mechanism of M2AH mediated viral budding has been shown to involve membrane bending when M2AH is integrated into the phospholipid membrane contains cholesterol.

With the desire to express recombinant protein that meet pharmaceutical demand or biomedical study needs, various recombinant protein expression systems have been developed. From most simple but high productivity bacterial expression system to the most complex but delicate mammalian cell expression system, different expression system provide unique niche in term of post-translational modifications. Cell free protein expression system like wheat germ extract, rabbit reticulocyte lysate or E.coli extract system provides biomedical researchers a useful tool that enables high-throughput functional genomic and proteomics.

### SUMMARY OF INVENTION

This disclosure describes the creation of a novel reagent that may be used to produce self-assembled protein nanoparticle with several functions including stimulate long duration antibody response to a specific target peptide when the peptide is inserted into this nanoparticle through genetic recombination. This reagent is composed of two components, first, a polymerization module contains an amphipathic helical peptide derived from M2 protein of type A influenza virus and two point mutations that stabilized protein nanoparticle; second, an target peptide presentation module contains a superfolder green fluorescent protein (sfGFP) with an target peptide insertion site located between beta sheet 8 and 9 of sfGFP. An 8xHis tag may be located N-terminal to the insertion site to facilitate protein purification. The application of this reagent in various fields is started by synthesis of a small gene that contains the coding sequence of a target peptide. Then this gene is inserted into the target peptide insertion site through genetic recombination to create a protein expression plasmid. This plasmid is transformed into protein expressing E. coil strain and cultivated for protein induction. The recombinant protein expressed will assembled spontaneously into SAPN post translation and can be purified using Ni-NTA resin or other methods. The purified protein can then be used for different applications, for example: immunizing animal for high affinity antibody production or directly as vaccine against infectious diseases.

### BRIEF DESCRIPTION OF DRAWING

Advantages of the present invention will become apparent to those skilled in the art with the benefit of following detailed description of embodiments and reference to the accompanying drawings in which:

FIG. 1: Depicts the primary structure of the recombinant self-assembled protein of current invention. This recombinant protein contains two modules, a polymerization module and a target peptide presentation module. The polymerization module contains an amphipathic helical peptide (LYRRLE) with a peptide sequence of MDRLFFKCLYRRLEYGLKRG. The target peptide presentation module contains a sfGFP with a target peptide insertion site located between beta sheet 8 and 9. The 8xHis tag for recombinant protein purification may located in front of the target peptide insertion site or other site.

FIGS. 2A-D: Depicts the characterization of AH3-GFP mediated SAPN. A) The formation of higher order protein structure when GFP is fused to AH3 peptide. This GFP is derived from pEGFP-C1 vector with a primary sequence of enhanced GFP (EGFP). Protein preparations of His-GFP or AH3-GFP were centrifuged on size exclusion membrane with molecular weight cut off (MWCO) of 100 kDa, 300 kDa and 1000 kDa. Protein passed through the membrane were analyzed by SDS-PAGE for comparison with input protein. B) Transmission electronic microscope images of AH3-GFP is shown. The scale bar is 100 nm. TEM images of AH3-GFP protein complex was obtained after the negative staining with phosphotungstic acid. Images were taken using Tecnai G2 Spirit Twin. C) Immunization of mice once with His-GFP and AH3-GFP protein without deoxycholate (DOC) is followed for 6 months. Proteins mixed with deoxycholate were only observed up to the first month. Sera collected were analyzed by ELISA against GFP to identify anti-GFP IgG geometric mean titer (GMT). D) The model of AH3-GFP polymer structure is constructed according to TEM images.

FIGS. 3A-D: Depicts the AH3 variants generated and the identification of AH3 variants that enable formation of hydrophobic patch free SAPN. A) A list of AH3 variants that were generated through protein structural model guided mutagenesis. The amino acids altered from AH3 in AH3-sfGFP-2xhM2e are underlined. B) Evaluating the applicability of using step sucrose gradient to identify hydrophobic patch free SAPN formed by AH3 variant. In a 13-ml polypropylene tube (Beckman cat #14287), the bottom was layered first with 1 ml 85% (w/v) sucrose solution and topped with 2 ml 45% (w/v) sucrose solution and then 7 ml 15% (w/v) sucrose solution. Sudan III stock solution was prepared as 0.5% in isopropanol. The tube 1 is a control centrifuge tube layered with sucrose step gradients but without adding bacterial lysate. The tube 2 was the centrifuge tube layered with bacterial lysate together with Sudan III solution. Baterial lysate was prepared by centrifuge the sonicated bacterial cell by 10K rpm for 10 minutes in a SS34 rotor by Sorvall 6C. After layered the bacterial lysate with Sudan III on sucrose step gradients, tube were centrifuged at 35K rpm in a SW41Ti rotor for 2 hours. After centrifugation, tube 1 Sudan III dye migrated and located in the junction between 45% and 85% sucrose, but in tube 2, Sudan III dye stop at the junction between 15% and 45% sucrose solution. This data supporting the accumulation of bacterial membrane and associated Sudan III dye in 15% to 45% sucrose junction when bacterial lysate is added. C) Using the same sucrose step gradient, one milliliter of bacterial lysate contains recombinant proteins of different AH3 variants were layered on top of sucrose step gradient and centrifuged at 35K rpm in a SW41Ti rotor for 2 hours, the centrifuge tube were then imaged for the distribution of fluorescent SAPN by exposed to a LED light bulb emitting 450 nm wavelength light. The arrangement of AH3 variants: tube 1, AH3 (Sequence 3); tube 2, LY (Sequence 4); tube 3, LYRLLK (Sequence 5); tube 4, LYRLLE (Sequence 6); tube 5, LYRRLE (Sequence 7); tube 6, RRLE (Sequence 8); tube 7, RRLD (Sequence 9). The result shows SAPN formed by LYRRLE, RRLE and RRLD variants still form SAPN and sedimented to the junction between 45% and 85% sucrose fractions. And less protein co-sedimented with bacterial membrane. But most of SAPN formed by AH3, LY, LYRLLK or LYRLLE variants are co-sedimented with bacterial membrane and none reach 45%-85% junction. D) The TEM image of LYRRLE variant of AH3-sfGFP-2xhM2e is shown. Scale bar is 50 nm. These results suggesting LYRRLE, RRLE and RRLD variants are able to form SAPNs that are void of hydrophobic patch and do not interact with bacterial membrane.

FIGS. 4A-B: Depict the thermal stability of LYRRLE-sfGFP-2xhM2e SAPN. A) LYRRLE-sfGFP-2xhM2e and AH3-sfGFP-2xhM2e were desalted into 1/2xGF buffer and kept in rom temperature for 20 days. Proteins remain in solution is obtained by centrifugation of protein solution by 14.5K rpm for 5 minutes. Protein integrity is evaluated by electrophoresis in SDS-PAGE and visualized by coomassie blue staining. B) LYRRLE-sfGFP-2xhM2e SAPN was first desalted into 1/2xGF buffer and kept in 4° C. or 37° C. for 4 months. Mice were immunized once by 20 μg LYRRLE-sfGFP -2xhM2e in three preparations: freshly prepared protein (1 w), kept in 4° C. for 4 months (4-4 m) or 37° C. for 4 months (37-4m). Sera were collected at day 14 and then analyzed for anti-hM2e IgG geometric mean titers (GMT). The result shows there is no difference between 4° C. or 37° C. storage. These results support the stability and activity of LYRRLE-sfGFP based SAPN in high temperature storage. (N=5)

FIGS. 5A-C: Depicts the generation of long term immune response by hydrophobic patch free SAPN. Purified recombinant proteins of AH3-sfGFP-2xhM2e and LYRRLE-sfGFP-2xhM2e were desalted into 1/2xGF buffer (10 mM NaPO4, 150 mM NaCl, pH 7.4) and then mice were immunized at a single dose of 20 μg each. Control mice were immunized with 1/2xGF buffer (PBS). Blood were collected through facial vein prick at day 14, 50, 90 and 200 and sera were tested for anti-hM2e IgG titer after 100 folds dilution. The OD 450 reading of ELISA results were shown for all 5 mice in either A) PBS, B) AH3-sfGFP-2xhM2e or C) LYRRLE-sfGFP-2xhM2e group. The result shows that four out of five mice immunized with LYRRLE variants SAPN has long lasting antibody response whereas only one out of five mice immunized with original AH3 based SAPN has long lasting antibody response.

FIGS. 6A-B: Depicts the generation of high affinity antibody using LYRRLE-sfGFP based SAPN. Recombinant protein LYRRLE-sfGFP-2xhM2e was expressed and purified using Ni-NTA resin and desalted to 1/2xGF buffer. Mice were immunized twice with 14 days interval using muscular injection at 20 μg protein each. Serum (#830) was collected at day 90 post immunization and used for detecting immunizing recombinant protein by western blot (WB). A) Three different proteins were used as substrates in WB, 1) His-sfGFP with insertion site and N-terminal His tag but no AH3 peptide, 2) LYRRLE-sfGFP-2xhM2e, 3) LYRRLE-sfGFP-2xM2e with different M2e sequence than sample 2. Control antibody is anti-His mAb purchased from Sigma (Sigma, cat # 70796-m). VP-10 is a serum collected from mice immunized with AH3-sfGFP-M2e protein. B) Testing the antibody (#830) affinity against immunizing antigen (LYRRLE-sfGFP-2xhM2e) in different amount: 10 ng, 5 ng, 2ng, 1 ng and 0.2 ng. Control antibody is an anti-His mAb from Sigma. The result show that LYRRLE-sfGFP-2xhM2e immunization activates antibody that is both of high affinity and high specificity.

FIGS. 7A-B: Depicts the generation of CMTR2 peptide antibody using LYRRLE-sfGFP based SAPN. A peptide sequence from mouse CMTR2 ORF was selected for gene synthesis and inserted into LYRRLE-sfGFP SAPN through genetic recombination to generate LYRRLE-sfGFP-CMTR2. Recombinant protein was expressed and purified for mice immunization in the absence of adjuvant. After 4 immunizations, sera were collected from mice and used for western blot in 1:1000 dilution to detect recombinant protein at 1 ng scale. A) SDS-PAGE of individual proteins generated using same LYRRLE-sfGFP platform. His-sfGFP (lane 1), LYRRLE-sfGFP-2xhM2e (lane 2) LYRRLE-sfGFP-CMTR2 (lane 3), LYRRLE-sfGFP-CMTR2-6 (lane 4), LYRRLE-sfGFP-CMTR2-B (lane 5), LYRRLE-sfGFP-Notch3-1 (lane 6), LYRRLE-sfGFP-Notch3-3 (lane 7). B) Protein listed in panel A were mixed and adjusted to 1 ng each for WB analysis using sera collected from mice immunized with LYRRLE-sfGFP-CMTR2. H stands for anti-His mAb from Sigma. The data shows the sera from all five mice (66-70) are able to detect CMTR2 peptide containing recombinant protein at 1 ng scale with high intensity.

FIGS. 8A-B: Depicts the depletion of anti-sfGFP antibody by an affinity resin. A serum sample that collected from mouse immunized with LYRRLE-sfGFP-2xhM2e twice were used in an antibody depletion efficiency test. Ten microliter of serum was first diluted 20 folds by ELISA blocking buffer (10 mM NaPO4, 150 mM NaCl, 0.05% Tween 20, 1% BSA pH 7.4) and passed through either 50 microliter or 100 microliter of affinity resin. Affinity resin is conjugated with His-sfGFP in a density of 2 mg/ml. Flow through were then analyzed by ELISA to detect both A) anti-His-sfGFP IgG and B) anti-hM2e IgG. The result shows that the sfGFP affinity resin is able to remove up to 90% anti-sfGFP IgG by 50 μl resin or up to 98% anti-sfGFP IgG by 100 μl resin. But the affinity resin has little effect on anti-hM2e IgG titer.

While the invention may be susceptible to various modifications and alternative forms, specific embodiments thereof are shown by ways of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictate otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected. The term “target peptide” means a peptide sequence either served as antigen, diagnostic probe, or protein binding peptide.

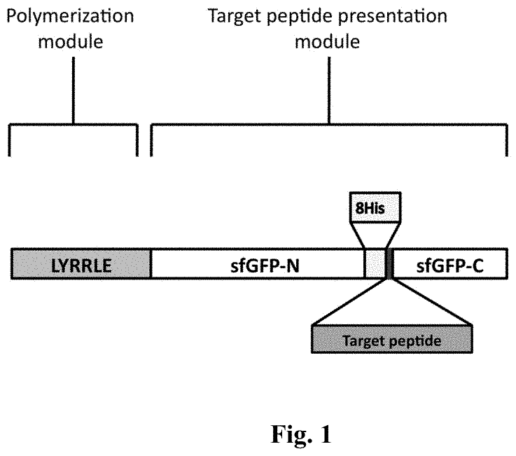

Our subject matter comprises a recombinant protein that self-assembled into protein nanoparticle (SAPN) that may incorporates target peptides on the surface through genetic recombination. In one preferred embodiment, a fusion protein composed of two parts: a polymerization module composed of an amphipathic helical peptide modified from M2 protein of type A influenza virus; a target peptide presentation module composed of a superfolder GFP and a short DNA fragment inserted in frame in the loop between beta sheet 8 and 9. That short DNA fragment may encodes an 8xHis tag and a target peptide insertion site (FIG. 1, Sequence 1).

In another embodiment, the sfGFP in target peptide presentation module may replaced by a sfmCherry or a Thermal green protein (TGP) for target peptide incorporation.

In another embodiment, the sfGFP of target peptide presentation module maybe replaced by another fluorescent protein with beta barrel structure and composed of 11 beta sheet and 1 alpha helix that emits fluorescent when excited by a photon.

In another embodiment, the polymerization module may be fused to the C-terminal of target peptide presentation module.

In another embodiment, the polymerization module may contains peptide sequence from amino acid 44 to amino acid 62 of type A influenza virus M2 protein.

In one embodiment, the LYRRLE peptide (sequence no. 7) in the polymerization module may be replaced by a peptide with sequence of DRLFFKCLYRRLDYGLKRG.

In another embodiment, the LYRRLE peptide (sequence no. 7) in the polymerization module may be replaced by peptide contains sequence no. 8.

In another embodiment, the LYRRLE peptide (sequence no. 7) in the polymerization module may be replaced by peptide contains sequence no. 9.

In another embodiment, the polymerization module may comprise a peptide with sequence of LFFKCLYRRLEYGL (sequence 12).

In one embodiment, the target peptide may be a tumor antigen that regulate tumor growth. The SAPN incorporated with tumor antigen may be used as therapeutic vaccine against tumor by immunizing human with tumor.

In one embodiment, the target peptide may be a protein of infectious pathogen that mediates infection process.

In one embodiment, the target peptide may be a virus receptor on human cell.

In one embodiment, the target peptide may be a tumor binding peptide that enables concentration of fluorescent SAPN onto the tumor site when SAPN is injected into a host.

In one embodiment, the target peptide may be a streptavidin binding peptide that enable the LYRRLE-sfGFP based SAPN to crosslink with large biotinylated protein.

In one embodiment, the stability of LYRRLE-sfGFP based SAPN may be enhanced by adding a 6His tag to the N-terminal of recombinant protein; to the C-terminal of recombinant protein.

In one embodiment, the stability of LYRRLE-sfGFP based SAPN may be enhanced by adding disaccharide like Trehalose or Sucrose.

In another embodiment, LYRRLE-sfGFP based SAPN may attached a signal peptide to its N-terminal to facilitate export of SAPN into the periplasmic space of E.coli. These peptides include signal peptides from maltose binding protein (MBP), beta-lactamase, Cry1Ia toxin, PelB, HlyA, GeneIII.

In the preferred embodiment, the production of high affinity antibody is started by synthesis of a gene coding target peptide using chemical synthesis or PCR. This gene can then be cloned into target peptide insertion site through DNA recombination techniques. The result protein expression plasmid can then be transformed into protein expression E. coli strain like ClearColi BL21(DE3) followed by protein induction using IPTG in a low temperature (20° C.) incubator to enhance protein folding and productivity. The expressed protein can then be purified using Ni-NTA resin after bacteria homogenized by sonication in an ultrasonic device (Misonix sonicator 3000). The recombinant protein containing bacterial lysate was then centrifuged at 10,000 rpm for 10 minutes in a SS34 rotor in Sorvall RC6 centrifuge to remove cell debris. Recombinant protein is then purified by binding to Ni-NTA resin and eluted by Elution buffer containing 500 mM imidazole. The recombinant protein expressed and purified following above mentioned procedures forms SAPN spontaneously without further process and maybe stored in Elution buffer (20 mM NaPO4, 300 mM NaCl, 500 mM Imidazole pH 8.0) for several months in 4° C. Before immunization, the protein is desalted into 1/2xGF buffer with pH ranged between 7.0 and 8.0 using desalting column (GE illustra NAP-5). The protein may injected directly without adjuvant into the hind limb of mice for immunization and blood were collected 2 weeks or later after immunization for ELISA assay.

In one embodiment, this SAPN may be used in immunizing animals other than mouse such as fish, rabbit, chicken, canine, feline, swine, cattle, horse and human.

In one embodiment, the recombinant protein may be produced in other nonbacterial expression systems such as yeast, insect cells, plants, and mammalian cell cultures.

In one embodiment, the the DNA fragment coding LYRRLE-sfGFP based SAPN may be translated in a cell free protein expression system to generate large number of SAPN clones that each incorporate a different target peptide.

In one embodiment, LYRRLE-sfGFP based SAPN may mixes with adjuvants to enhance antigenicity of target peptide before immunization.

In another embodiment, the polymerization module may fused with another protein directly and forms a completely new SAPN with new functions. For example, LYRRLE peptide may be fused with a single chain variable fragment (scFv) and drive the formation of a scFv based SAPN with ability to bind multiple scFv targets in the same time for higher affinity.

In another embodiment, the polymerization module may fused directly to a GFP antibody to form a SAPN covered with target protein binding GFP antibody that served as diagnostic tool.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred mode for its practice. However, those of skill in the art should, inlight of the present disclosure, appreciate that many changes can be made in the specific embodiments which are disclosed and still obtain a like or similar result without departing from the spirit and scope of the invention.

### Example 1

Fusion of AH3 with GFP enables nanoparticle formation and long term immune response.

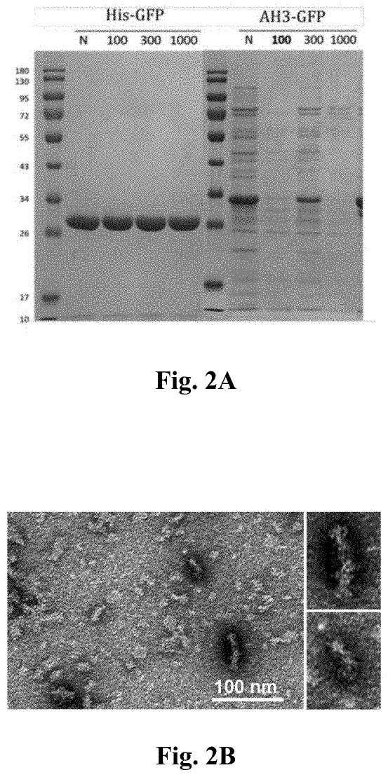

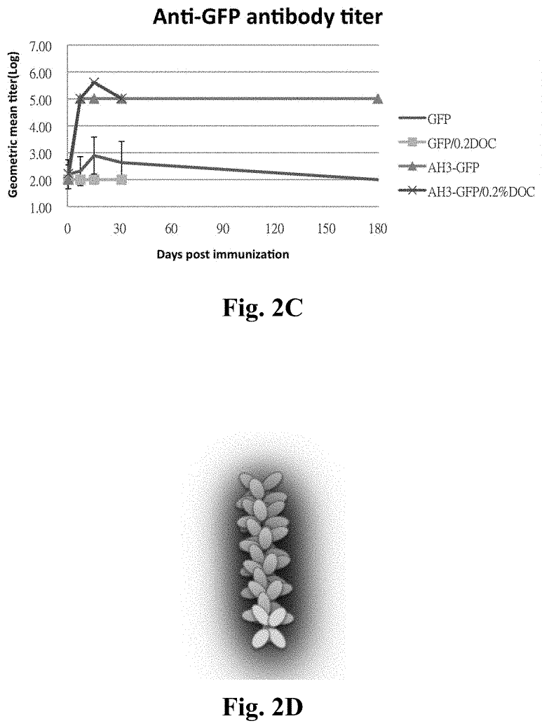

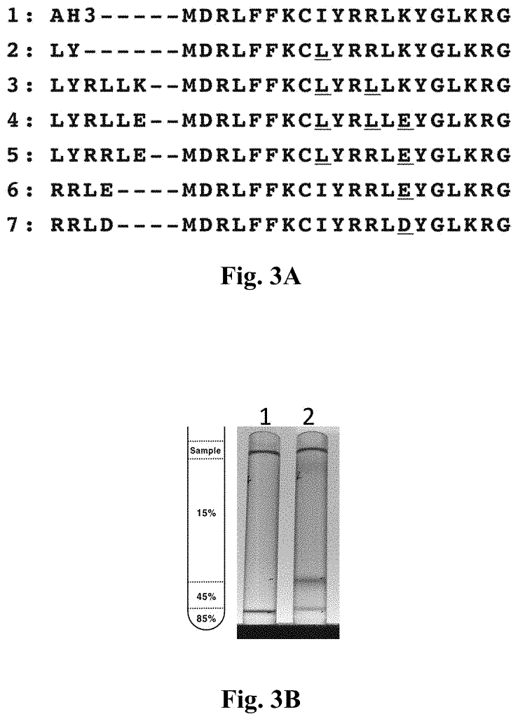

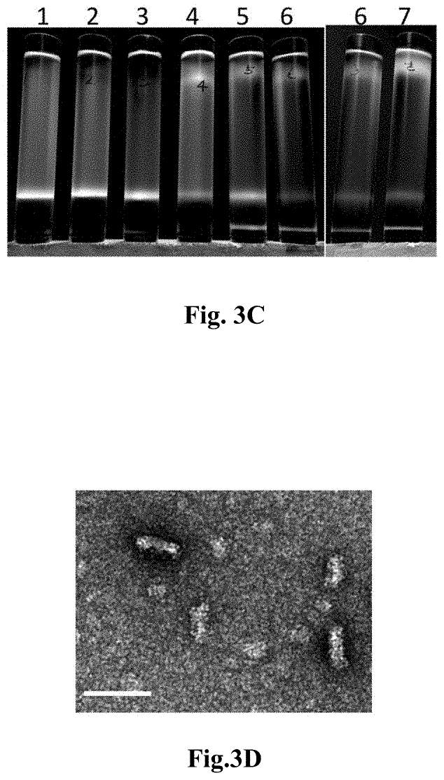

AH3 is an amphipathic helical peptide derived from M2 protein of type A influenza virus. GFP is cloned from pEGFP-C1 vector that encode enhanced green fluorescent protein. Compare to His-GFP, AH3-GFP forms higher order protein structure that has a molecular weight larger than 1000 kDa as shown by centrifugation of AH3-GFP protein solution in size exclusion membrane of different MWCO (FIG. 2A). When examined under transmission electronic microscope, AH3-GFP is forming a rod shape structure (FIG. 2B). This data support the formation of AH3-GFP polymer that larger than 1000 kDa. When the AH3-GFP recombinant protein is used in mice immunization, it induced anti-GFP antibody that lasting for 6 months (FIG. 2C). The structure of AH3-GFP polymer is predicted by protein modeling using AH3 peptide in a-helix structure. The predicted structure from protein modeling shows the AH3 peptide served as a polymerization center that stack AH3-GFP tetramer in the shape of a cross on top of another tetramer and forms rod like structure (FIG. 2D). Each predicted tetramer contains a hydrophobic patch that drives the polymerization process. As shown in FIG. 3, AH3-sfGFP-2xhM2e protein interact with bacterial membrane and co-sedimented with bacterial membrane. After protein model guided mutagenesis, variants of AH3 peptide sequence were introduced into the AH3-sfGFP-2xhM2e fusion protein and the removal of hydrophobic patch by mutagenesis was confirmed by analyzing bacteria lysates containing SAPN with AH3 variants in sucrose step gradient assay. The result shown that AH3 variants, LYRRLE (sequence no. 7), RRLD (sequence no. 9) and RRLE (sequence no 8) has decreased membrane co-sedimentation and it suggests the removal of hydrophobic patch by these variants. This data in FIG. 3 suggest replacing the Lysine in position 13 with either Glutamic acid (E) or Aspartic acid (D) is important to remove hydrophobic patch and may contribute to particle stability. One AH3 variant, LYRRLE is found to maintain rod shape structure under TEM (FIG. 3C and FIG. 3D).

### Example 2

Evaluate the thermal stability of LYRRLE-sfGFP based SAPN.

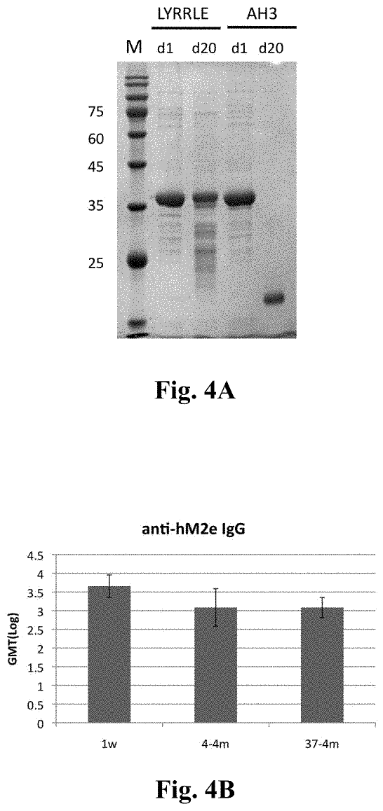

Fluorescent protein is known to endure high temperature up to 75° C., or even 85° C. for superfolder GFP without losing fluorescent, an indication of structural unfolding. The ability of remaining active during high temperature storage will enable the development of a vaccine that can reach remote area beyond cold-chain. The stability of LYRRLE variant after removing the hydrophobic patch from SAPN was evaluated in physiological buffer. AH3-sfGFP-2xhM2e and LYRRLE-sfGFP-2xhM2e were desalted into physiological buffer (10 mM NaPO4, 150 mM NaCl, pH 7.4) and adjusted to 1 mg/ml then stored in RT (25° C.) for 20 days. On day 10, the AH3-sfGFP-2xhM2e become cloudy but not the LYRRLE-sfGFP-2xhM2e. On day 20, the aggregated protein was removed by centrifugation at 14.5 krpm for 5 minutes in a microfuge. The supernatants were used in SDS-PAGE analysis for protein integrity. The result shows the mutation of I8L and K13E stabilized AH-sfGFP based SAPN (FIG. 4A). To test whether LYRRLE-sfGFP based SAPN is able to endure high temperature during storage, LYRRLE-sfGFP-2xhM2e is stored in physiological buffer at either 4° C. or 37° C. for 4 months. Then the proteins were used in mice immunization to test the thermal stability of this protein. Mice were divided into three groups, each immunized with 20 μg protein once with protein either stored in 4° C. or 37° C. for 4 months or freshly prepared protein. Sera were collected 14 days post immunization and used for ELISA assay detecting anti-hM2e peptide IgG titer. The result shows the 4 months storage of LYRRLE-sfGFP-2xhM2e protein in either 4° C. or 37° C. both decrease the activity to stimulate anti-hM2e IgG production by 4 folds when compared to fresh prepared protein (FIG. 4B). So the high temperature storage has no effect on LYRRLE-sfGFP based SAPN activity.

### Example 3

Construction, expression and immunization of a LYRRLE-sfGFP based SAPN presenting a broad spectrum influenza vaccine epitope, hM2e.

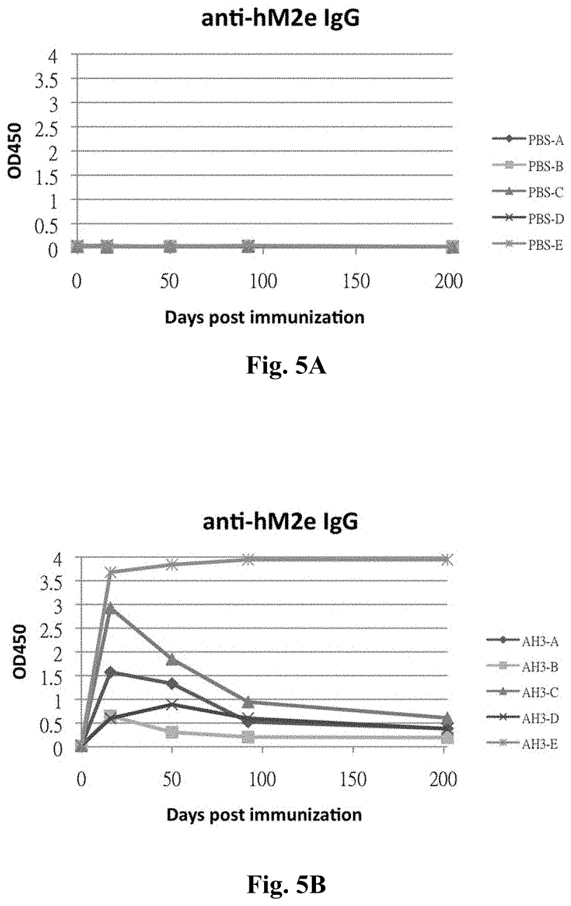

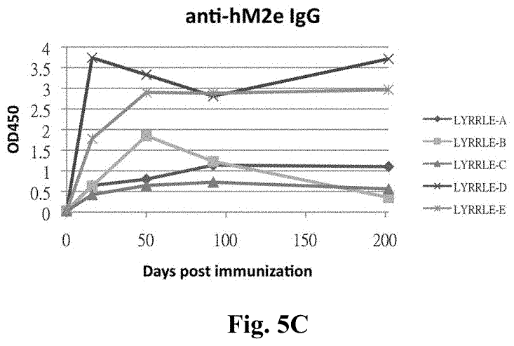

The gene encoding 2 copies of M2 ectopic domain (hM2e) from type A influenza virus strain, PR8, that separated by a linker was synthesized and inserted in frame into the insertion site on plasmid encoding a LYRRLE-sfGFP recombinant protein using genetic recombination. The insertion site is located in the loop between beta sheet 8 and 9 behind a 8His tag. The plasmid contains correct insertion (LYRRLE-sfGFP-2xhM2e) was transformed in ClearColi BL21(DE3) competent cell and plated on an agar plate with 50 μg/ml kanamycin. The plate was cultured in 37° C. incubator for 2 days. In the morning of third day, colonies are scraped from plate and resuspended in LB broth contain 50 μg/ml kanamycin. The broth is then shaked in 37° C. incubator until the O.D. 600 reach 0.5-0.7, then the culture flask is removed from incubator and temperature cooled down by placing it on ice. After adding 0.2 mM IPTG, the culture is placed in a incubator for protein induction at 20° C. for 16 hrs. The bacteria are harvested by centrifugation at 5000 rpm for 10 minutes in a SLA1500 rotor by RC6 centrifuge. Bacteria pellet was then resuspended in lysis buffer (20 mM NaPO4, 300 mM NaCl, 10 mM imidazole, pH 8.0) and sonicated in ice bath using an ultrasonic breaker (Misonix sonicator 3000) at 10 second on/20 second off cycles for 5 minutes in icy water. Insoluble debris was removed by centrifugation in 10000 rpm for 10 minutes using a Sorval SS34 rotor at 4° C. Soluble fraction containing the target protein was then used for purification by Ni-NTA resin as described in the user manual. Bound protein is eluted using a Elution buffer (20 mM NaPO4, 300 mM NaCl, 500 mM imidazole, pH 8.0). Eluted protein can be stored in 4° C. for extended period of time. Before mice immunization, protein is desalted using sephadex 25 column (GE illustra NAPS) into 1/2xGF buffer (10 mM NaPO4, 150 mM NaCl, pH 7.4). Immunization is carried out by injecting 20 μg of the recombinant protein, AH3-sfGFP-2xhM2e or LYRRLE-sfGFP-2xhM2e, into the hind limb muscle of mice once. Sera is collected after bleeding through facial vein at day 15, day 50, day 90 and day 202 for ELISA analysis to determine the anti-hM2e IgG titer (FIG. 5). The result shows the immunization of mice using LYRRLE-sfGFP-2xhM2e protein stimulated extended duration of anti-hM2e IgG titer in 4 out of 5 mice, but when immunizing mice using AH3-sfGFP-2xM2e protein, only 1 out of 5 mice has long lasting anti-hM2e IgG titer.

### Example 4

Detection and differentiating two M2e peptide variants using sera from mice immunized by LYRRLE-sfGFP-2xM2e and LYRRLE-sfGFP-2xhM2e proteins.

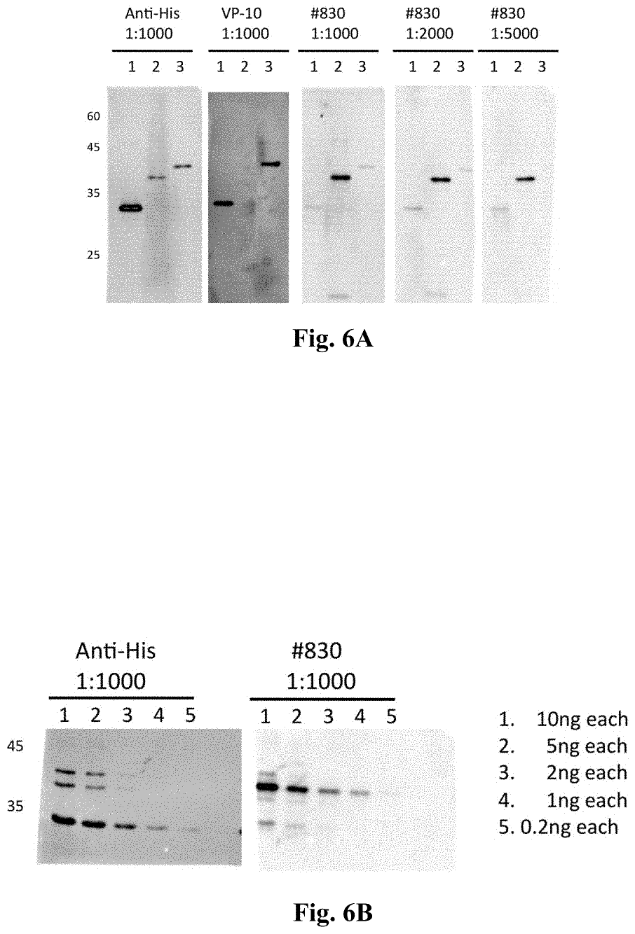

Three proteins, His-sfGFP (lane 1), LYRRLE-sfGFP-2xhM2e (lane 2) and LYRRLE-sfGFP-2xM2e (lane 3) are loaded individually in SDS-PAGE gel and blotted on PVDF membrane after electrophoresis. Membrane is then probed with sera VP-10 (immunized with M2e containing SAPN) or #830 (immunized with hM2e containing SAPN) and detected by ECL reagent. The result show the sera VP-10 or #830 only detect protein with same peptide sequence of antigen with five amino acid difference between hM2e (SLLTEVETPIRNEWGSRSNGSSD 23 a.a.) and M2e (SLLTEVETPTRSEWESRSSDSSD 23 a.a.) (FIG. 6A). Also #830 can detect 10 ng protein after dilute 1:5000. Note: weak detection of lane 1 and lane 3 by #830 maybe due to the induction of anti-His antibody. The sera #830 also has high affinity to hM2e peptide, it can detect as little as 1 ng recombinant protein in WB (FIG. 6B).

### Example 5

Construction, purification and immunization of LYRRLE-sfGFP-CMTR2 SAPN.

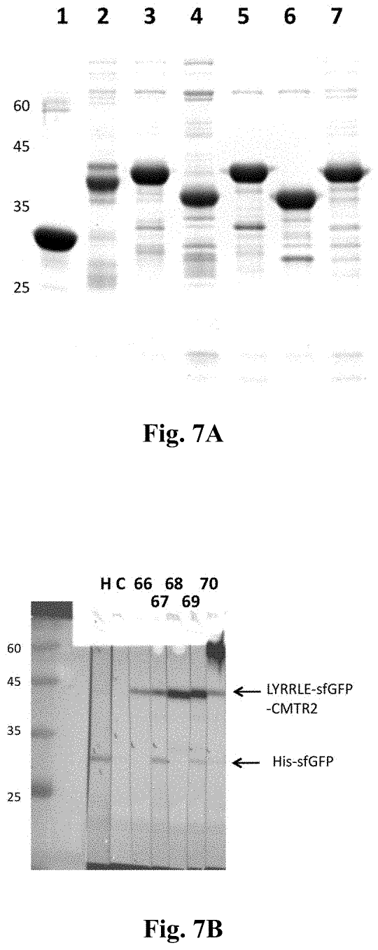

A gene (Sequence 10) encoding 2 tandem copies of CMTR2 (Cap Methyltransferase 2) peptide (a.a 233-253) was synthesized and constructed into the insertion site of LYRRLE-sfGFP expression plasmid by genetic recombination. The constructed plasmid, LYRRLE-sfGFP-CMTR2 was transformed into ClearColi BL21(DE3) competent cell and plated on kanamycin plate. Protein expression is induced by 0.2 mM IPTG in 20° C. for 16 hrs after the optical density of bacterial culture reach O.D. 600 between 0.5-0.7. Recombinant protein is purified using Ni-NTA resin after bacterial lysate is clearified by centrifugation. Recombinant protein bound to Ni-NTA resin was eluted in Elution buffer that contains 500 mM Imidazole. LYRRLE-sfGFP-CMTR2 desalted into physiological buffer is used for immunization. Following 4 consecutive immunization of 40 μg with 14 days interval, sera were prepared from blood collected from mouse facial vein and used for western blot analysis. A) Antigen proteins present in the mixture contains including His-sfGFP (lane 1), LYRRLE-sfGFP-2xhM2e (lane 2) LYRRLE-sfGFP-CMTR2 (lane 3), LYRRLE-sfGFP-CMTR2-6 (lane 4), LYRRLE-sfGFP-CMTR2-B (lane 5), LYRRLE-sfGFP-Notch3-1 (lane 6), LYRRLE-sfGFP-Notch3-3 (lane 7). B) The protein mixture contains 1 ng of each proteins shown in panel A and was separated by SDS-PAGE and blotted on to PVDF membrane and probed with 5 sera generated using LYRRLE-sfGFP-CMTR2. The result shows sera from mice immunized by LYRRLE-sfGFP-CMTR2 only detect proteins with corresponding inserted peptide but not the other proteins share same LYRRLE-sfGFP sequence (FIG. 7).

### Example 6

Depletion of anti-sfGFP IgG from immune sera using an affinity resin.

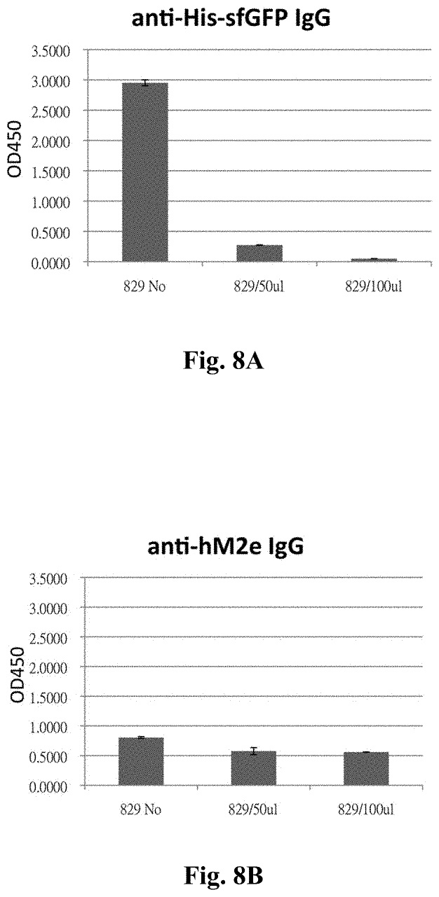

The recombinant protein sfGFP-8His without polymerization module or target peptide is expressed and purified using Ni-NTA resin. Eluted protein is first adjusted to 4 mg/ml and then 2 ml sfGFP-8His protein is first desalted into coupling buffer (0.6M NaCitrate, 0.1M MOPS pH 7.5) and then followed by mixing with 0.5 gram of activated beads (Pierce Ultralink Biosupport) for crosslinking. The coupling reaction mixture is rotated top-to-bottom continuously for 1 hr and then free protein removed. The unreacted crosslinker was quenched by 5 ml of 1M Ethanolamine by rotating for 3 hrs. The His-sfGFP protein coated bead is then loaded into an empty chromatography column and beads washed by 10 bed volume of 1M NaCl and 10 bed volume of sterilized Milli-Q water to remove non-covalent linked His-sfGFP protein. The affinity resins prepared following this protocol are stored in 20% Ethanol in 4° C. To test the ability of His-sfGFP resin in removing background anti-sfGFP IgG and anti-His IgG, 10 μl sera from mouse immunized twice with LYRRLE-sfGFP-2xhM2e antigen is first diluted 20 folds in ELISA blocking buffer (1% BSA, 10 mM NaPO4, 150 mM NaCl, 0.05% Tween-20 pH 7.5) and then passed through small columns contain either 50 μl or 100 μl affinity resin for three times. Flow throughs were used for ELISA against either hM2e peptide or sfGFP-8His protein coated on microplate. The result shows result of OD450 using sera with final dilution of 400 folds. It shows the sfGFP affinity resin can effectively remove anti-sfGFP IgG but not anti-hM2e IgG (FIG. 8).

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taked as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

A request to make an amendment to the sequence listing was made at Feb. 16, 2022. The new sequence listing file is “VAX _20200914_SEQ_rev.txt”, created at Feb. 14, 2022 with a file size of 9 KB.

What claimed is:

1. A recombinant self-assembled protein, comprising a polymerization module and a target peptide presentation module;
the polymerization module is fused in frame to the target peptide presentation module through genetic recombination;
polymerized into hydrophobic patch free self-assembled protein nanoparticle when expressed in a host.

2. The recombinant self-assembled protein of claim 1, whereas the polymerization module comprising a peptide with sequence between amino acids 44 to amino acid 62 of M2 protein from type A influenza virus.

3. The recombinant self-assembled protein of claim 1, whereas the polymerization module comprising a peptide with sequence of DRLFFKCLYRRLXYGLKRG, wherein X is a group contains Glutamic acid (E) and Aspartic acid (D).

4. The recombinant self-assembled protein of claim 1, whereas the polymerization module comprising a peptide with sequence of DRLFFKCIYRRLXYGLKRG, wherein X is a group contains Glutamic acid (E) and Aspartic acid (D).

5. The recombinant self-assembled protein of claim 1, whereas the target peptide presentation module comprising a fluorescent protein contains a target peptide insertion site between beta sheet 8 and 9.

6. The recombinant self-assembled protein of claim 5, whereas the fluorescent protein comprising a protein with following features: (a) has a beta barrel structure composed of 11 beta sheets and 1 alpha helix; (b) it emits fluorescence upon excitation by a photon.

7. The recombinant self-assembled protein of claim 5, whereas the fluorescent protein comprising a superfolder green fluorescent protein, thermal green protein or superfolder mCherry protein.

8. The recombinant self-assembled protein of claim 5, whereas the peptide insertion site comprising an 8xHis tag and a target peptide.

9. The recombinant self-assembled protein of claim 8, whereas the target peptide comprising an antigen peptide.

10. The recombinant self-assembled protein of claim 1 when self-assembled into protein nanoparticle may serves as vaccine formulation.

11. A method for making high affinity antibody comprising the steps of; (a) designing a gene encoding a target peptide; (b) inserting it into the target peptide insertion site of recombinant self-assembled protein described in claim 1 through genetic recombination to make the recombinant protein expression vector; (c) transforming the recombinant protein expression vector into a protein expressing host and expressing and purifying the self-assembled protein nanoparticle comprising target peptide; (d) immunizing animal using this self-assembled protein nanoparticle without adjuvant; (e) collecting antibody by bleeding animal or generating monoclonal antibody against target peptide.

12. The method for making high affinity antibody of claim 11, whereas the protein expressing host comprising bacterial cells, yeast cells, insect cells, plant, mammalian cell cultures.

13. The method for making high affinity antibody of claim 11, whereas the protein expressing host comprising a cell free protein expression system.

14. A self-assembled peptide with a protein sequence of DRLFFKCLYRRLXYGLKRG, wherein X is a group contains Glutamic acid (E) and Aspartic acid (D).

15. A self-assembled peptide with a protein sequence of DRLFFKCIYRRLXYGLKRG, wherein X is a group contains Glutamic acid (E) and Aspartic acid (D).


SEQUENCE LISTING (12 sequences)

SEQ ID NO: 1 - protein, 279 aa, Artificial Sequence
  Synthesized
MDRLFFKCLYRRLEYGLKRGGTTSDVMSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSHHHHHHHHKLSELGSVQLADHYQQNTPIGDGPVLLPDNHYLSTQSVLSKDPNEKRDHMVLLEFVTAAGITHGMDELYK

SEQ ID NO: 2 - protein, 328 aa, Artificial Sequence
  Synthesized
MDRLFFKCLYRRLEYGLKRGGTTSDVMSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSHHHHHHHHKLSLLTEVETPIRNEWGSRSNGSSDSSGGSLLTEVETPIRNEWGSRSNGSSDELGSVQLADHYQQNTPIGDGPVLLPDNHYLSTQSVLSKDPNEKRDHMVLLEFVTAAGITHGMDELYK

SEQ ID NO: 3 - protein, 19 aa, Influenza A virus
  ref <308>: DATABASE ACCESSION NUMBER: Biorxiv/doi: https://doi.org/10.1101/2020.09.16.299149
  ref <309>: DATABASE ENTRY DATE: 2020-09-18
  ref <313>: RELEVANT RESIDUES IN SEQ ID NO: (1)..(19)
DRLFFKCIYRRLKYGLKRG

SEQ ID NO: 4 - protein, 19 aa, Artificial Sequence
  Synthesized
  ref <308>: DATABASE ACCESSION NUMBER: Biorxiv/doi: https://doi.org/10.1101/2020.09.16.299149
  ref <309>: DATABASE ENTRY DATE: 2020-09-18
  ref <313>: RELEVANT RESIDUES IN SEQ ID NO: (1)..(19)
DRLFFKCLYRRLKYGLKRG

SEQ ID NO: 5 - protein, 19 aa, Artificial Sequence
  Synthesized
  ref <308>: DATABASE ACCESSION NUMBER: Biorxiv/doi: https://doi.org/10.1101/2020.09.16.299149
  ref <309>: DATABASE ENTRY DATE: 2020-09-18
  ref <313>: RELEVANT RESIDUES IN SEQ ID NO: (1)..(19)
DRLFFKCLYRLLKYGLKRG

SEQ ID NO: 6 - protein, 19 aa, Artificial Sequence
  Synthesized
  ref <308>: DATABASE ACCESSION NUMBER: Biorxiv/doi: https://doi.org/10.1101/2020.09.16.299149
  ref <309>: DATABASE ENTRY DATE: 2020-09-18
  ref <313>: RELEVANT RESIDUES IN SEQ ID NO: (1)..(19)
DRLFFKCLYRLLKYGLKRG

SEQ ID NO: 7 - protein, 19 aa, Artificial Sequence
  Synthesized
  ref <308>: DATABASE ACCESSION NUMBER: Biorxiv/doi: https://doi.org/10.1101/2020.09.16.299149
  ref <309>: DATABASE ENTRY DATE: 2020-09-18
  ref <313>: RELEVANT RESIDUES IN SEQ ID NO: (1)..(19)
DRLFFKCLYRRLEYGLKRG

SEQ ID NO: 8 - protein, 19 aa, Artificial Sequence
  Synthesized
  ref <308>: DATABASE ACCESSION NUMBER: Biorxiv/doi: https://doi.org/10.1101/2020.09.16.299149
  ref <309>: DATABASE ENTRY DATE: 2020-09-18
  ref <313>: RELEVANT RESIDUES IN SEQ ID NO: (1)..(19)
DRLFFKCIYRRLEYGLKRG

SEQ ID NO: 9 - protein, 19 aa, Artificial Sequence
  Synthesized
DRLFFKCIYRRLDYGLKRG

SEQ ID NO: 10 - DNA, 207 bp, Homo sapiens
aagcttggcggcagcagcaccgcggatggcagctttgatagccagggcaacccgggcgaacaggaagcgctggtgagcagcctgcatggcggcagcagcaagcgcaagagcagcggcggcaccgcggatggcagctttgatagccagggcaacccgggcgaacaggaagcgctggtgagcagcctgcatggcggcagcagcgagctc

SEQ ID NO: 11 - protein, 19 aa, Artificial Sequence
  Synthesized
DRLFFKCLYRRLDYGLKRG

SEQ ID NO: 12 - protein, 14 aa, Artificial Sequence
  Synthesized
LFFKCLYRRLEYGL